(19) United States
(12) Patent Application Publication
(10) Pub. No.: US 2018/0289449 A1
(43) Pub. Date: Oct. 11, 2018

(54) COATED ORTHODONTIC APPLIANCES AND METHODS OF MAKING SAME

(71) Applicant: Ormco Corporation, Orange, CA (US)

(72) Inventors: Dwight P. Schnaitter, Salt Lake City, UT (US); Mark N. Coreil, Houma, LA (US); Vince Garcia, Chino Hills, CA (US)

(21) Appl. No.: 15/945,765

(22) Filed: Apr. 5, 2018

Related U.S. Application Data
(60) Provisional application No. 62/481,907, filed on Apr. 5, 2017.

(51) Int. Cl. A61C 7/14; A61L 27/10

(52) U.S. Cl. CPC A61C 7/146; B82Y 40/00; A61C 7/145; A61L 27/10

(57) ABSTRACT

An orthodontic appliance includes a nano-pillar coating on an external surface that is visible during use of the orthodontic appliance. The nano-pillar coating may absorb 99% of the incident visible light; has a pillar-like structure with pillar structures having a point that widens to a base; has a negative index of refraction at wavelengths in the visible light spectrum; and/or does not include nano rods. An orthodontic bracket includes a bracket body that includes an archwire slot, and a nano-pillar coating on an external surface of the bracket body. A movable member is movable between an opened position and a closed position, and the nano-pillar coating is on one or more external surfaces of the movable member. The bracket body is a ceramic and may be transparent or translucent. A method of making an orthodontic bracket includes forming a nano-pillar coating on at least a portion of the bracket body.

### CROSS REFERENCE TO RELATED APPLICATION

This application claims the benefit of U.S. Provisional Patent Application Ser. No. 62/481,907 filed on Apr. 5, 2017, the disclosure of which is expressly incorporated by reference herein in its entirety.

### TECHNICAL FIELD

The invention relates generally to orthodontic appliances, and more particularly to coated orthodontic brackets and methods of making those brackets.

### BACKGROUND

Orthodontic appliances, and in particular orthodontic brackets, represent a principal component of corrective orthodontic treatments devoted to improving a patient's occlusion. Conventional orthodontic brackets are ordinarily formed from stainless steel, which is strong, nonabsorbent, weldable, and relatively easy to form and machine. Patients undergoing orthodontic treatment using metal orthodontic brackets, however, may be embarrassed by the visibility of metal, which is not cosmetically pleasing. To improve the cosmetic appearance, certain conventional orthodontic brackets incorporate a bracket body of a transparent or translucent non-metallic material, such as a polymer resin or a ceramic, that assumes or mimics the color or shade of the underlying tooth. These appliances may be more appealing cosmetically, but have some functional drawbacks.

While forming traditional bracket bodies from transparent or translucent materials has generally improved the aesthetics of these brackets, additional aesthetic improvements have remained problematic. By way of example, current aesthetic self-ligating orthodontic brackets may use a transparent or translucent bracket body and closure member. Further, common materials used for orthodontic appliances, such as ceramic materials, have a proliferation of flat surfaces that reflect light, making the orthodontic brackets shiny and noticeable.

Other problems with orthodontic brackets of transparent or translucent materials may include a relatively high friction between many of the commonly used metal archwires and the material of the brackets, particularly those made of ceramic brackets. The high friction when combined with a large difference in hardness may ultimately result in notching of the archwire during orthodontic treatment. Both high friction and notching may increase treatment time because the bracket and tooth unit cannot move as easily relative to the archwire.

Additionally, self-ligating orthodontic brackets include a movable member to effectuate treatment by capturing the archwire within the bracket. The oral environment includes bacteria, known as dental plaque, and during treatment, plaque may form on surfaces of the orthodontic bracket that may interfere with movement of the movable member. Ultimately, the self-ligating feature of the bracket may malfunction and thus prevent operation of the movable member.

Consequently, while transparent or translucent orthodontic brackets have generally been successful, there is a need for improved, more fully aesthetic orthodontic brackets that overcome these and other deficiencies described above.

### SUMMARY

The present invention overcomes the foregoing and addresses other shortcomings and problems heretofore known for orthodontic appliances and methods of making those orthodontic appliances. In accordance with the principles of the present invention, an orthodontic appliance comprises a nano-pillar coating on one or more external surfaces that are visible during use of the orthodontic appliance.

In one embodiment, the nano-pillar coating is configured to absorb 99% of the incident visible light.

In one embodiment, the nano-pillar coating has a pillar-like structure that includes a plurality of pillar structures, each pillar structure including a point that widens to a base.

In one embodiment, the nano-pillar coating has a negative index of refraction at wavelengths in the visible light spectrum.

In one embodiment, the nano-pillar coating does not include nano rods.

According to one aspect of the present invention, an orthodontic bracket comprises a bracket body that includes an archwire slot and a nano-pillar coating on one or more external surfaces of the bracket body that are visible during use of the orthodontic bracket.

In one embodiment, the coating is configured to absorb 99% of the incident light.

In one embodiment, the coating has a pillar-like structure that includes a plurality of pillar structures, each pillar structure including a point that widens to a base.

In one embodiment, the coating has a negative index of refraction at wavelengths in the visible light spectrum.

In one embodiment, the nano-pillar coating does not include nano rods.

In one embodiment, a movable member is engaged with the bracket body and is movable between an opened position and a closed position, and the nano-pillar coating is on one or more external surfaces of the movable member.

In one embodiment, the bracket body is a ceramic material.

In one embodiment, the ceramic material is transparent or translucent.

According to one aspect, a method of making an orthodontic bracket including a bracket body having an archwire slot includes forming a nano-pillar coating on at least a portion of the bracket body.

In one embodiment, the orthodontic bracket further includes a movable member and wherein forming includes forming the nano-pillar coating on the movable member.

In one embodiment, a method of making the appliance includes injection molding the bracket body and forming the nano-pillar coating occurs after injection molding.

In one embodiment, a method of making the appliance includes injection molding the bracket body and forming the nano-pillar coating occurs during injection molding.

### BRIEF DESCRIPTION OF THE DRAWINGS

The accompanying drawings, which are incorporated in and constitute a part of this specification, illustrate embodiments of the invention and together with the detailed description given below, serve to explain various aspects of the invention.

FIG. 1 is a perspective view of a self-ligating orthodontic bracket in accordance with one embodiment of the invention, a ligating slide shown in the opened position.

FIG. 2 is a perspective view of the self-ligating orthodontic bracket shown in FIG. 1 with the ligating slide shown in the closed position.

FIG. 3 is a cross-sectional view of the self-ligating orthodontic bracket shown in FIG. 1 taken along the line 3-3.

FIG. 4 is a micrograph of an exemplary nano-pillar structure.

FIG. 5 is a perspective view of a twin tie-wing orthodontic bracket in accordance with one embodiment of the invention.

### DETAILED DESCRIPTION

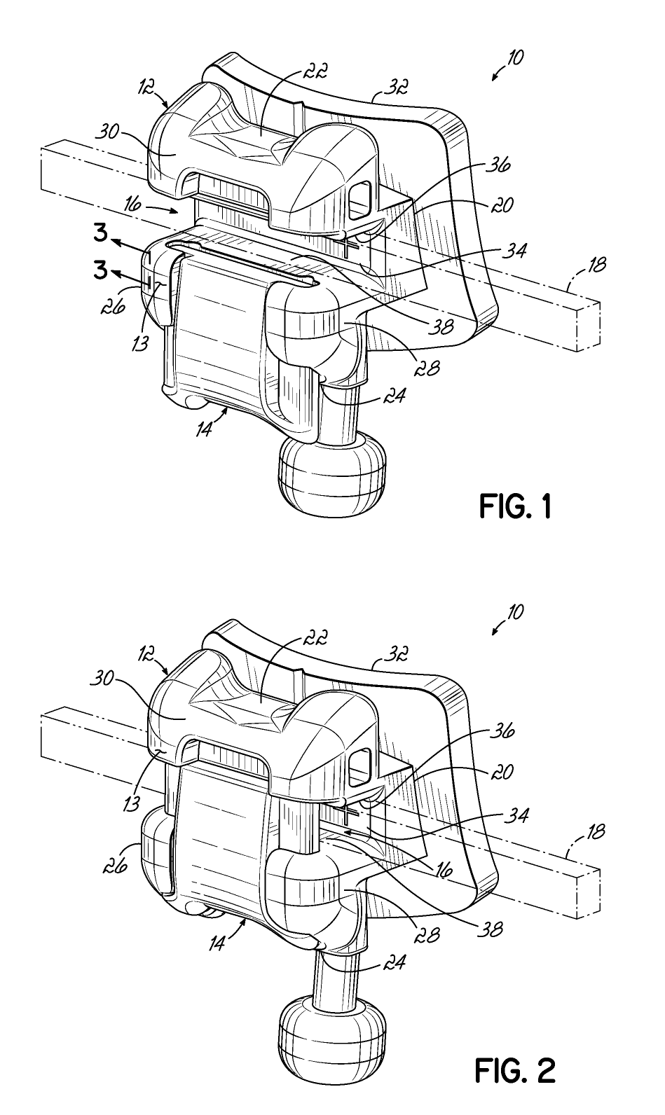

Referring now to the drawings, and to FIGS. 1-3 in particular, in one embodiment, the orthodontic appliance is an orthodontic bracket 10 and includes a bracket body 12. In the case of a self-ligating orthodontic bracket, the bracket 10 may include a movable closure member 14 coupled to the bracket body 12. A nano-pillar coating 13, described below, may cover all or a majority of the visible portions of the bracket body 12.

In the embodiment shown, the ligating slide 14 is slidably coupled with the bracket body 12. The bracket body 12 has an archwire slot 16 that receives an archwire 18 (shown in phantom) for applying corrective forces to the teeth during orthodontic treatment. The ligating slide 14 is movable relative to the archwire slot 16 between an opened position (FIG. 1) in which the archwire 18 is insertable into the archwire slot 16 and a closed position (FIG. 2) in which the archwire 18 is retained within the archwire slot 16. Moreover, while the movable closure member is described herein as a ligating slide, the invention is not so limited. Although not specifically shown in the figures, the movable closure member may include other movable structures (e.g., latch, spring clip, door, etc.) that are capable of moving from an opened position to a closed position.

The orthodontic bracket 10, unless otherwise indicated, is described herein using a reference frame attached to a labial surface of a tooth on the lower jaw. Consequently, as used herein, terms such as labial, lingual, mesial, distal, occlusal, and gingival used to describe the orthodontic bracket 10 are relative to the chosen reference frame. The embodiments of the invention, however, are not limited to the chosen reference frame and descriptive terms, as the orthodontic bracket 10 may be used on other teeth and in other orientations within the oral cavity. For example, the orthodontic bracket 10 may also be coupled to the lingual surface of the tooth and be within the scope of the invention. Those of ordinary skill in the art will recognize that the descriptive terms used herein may not directly apply when there is a change in reference frame. Nevertheless, embodiments of the invention are intended to be independent of location and orientation within the oral cavity and the relative terms used to describe embodiments of the orthodontic bracket are to merely provide a clear description of the embodiments in the drawings. As such, the relative terms labial, lingual, mesial, distal, occlusal, and gingival are in no way limiting the invention to a particular location or orientation.

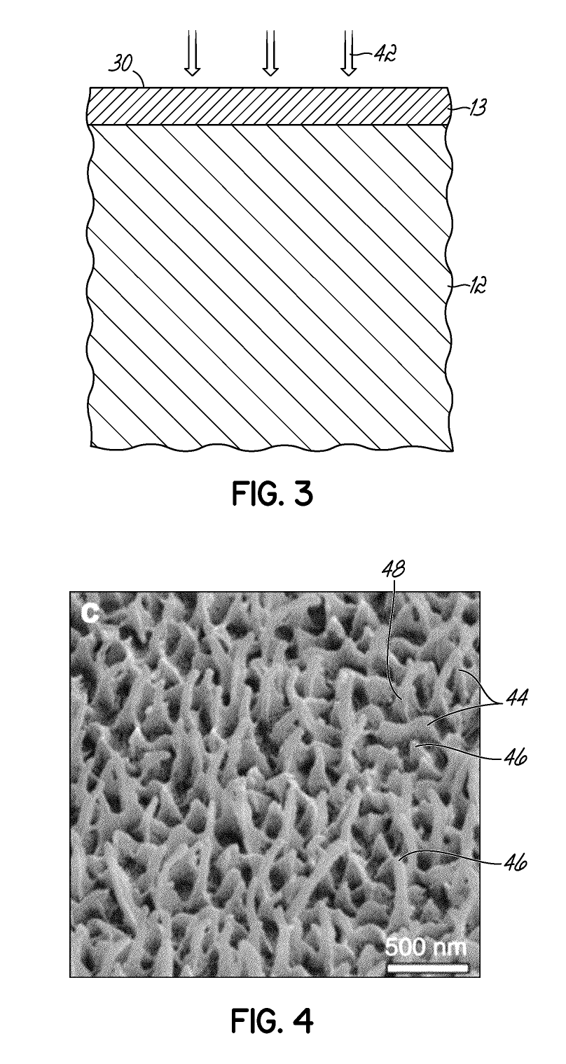

With reference to FIGS. 1 and 2, when mounted to the labial surface of a tooth carried on the patient's lower jaw, the bracket body 12 has external surfaces or sides including a lingual side 20, an occlusal side 22, a gingival side 24, a mesial side 26, a distal side 28 and a labial side 30. With reference to FIG. 2, in one embodiment, the coating 13 may be on any single one or a combination of the sides 20, 22, 24, 26, 28, 30. For example, the external surface of the labial side 30 may be defined by the coating 30. In that exemplary embodiment, the coating 30 defines the labial-most surface of the bracket body 12. Light that impinges upon the orthodontic bracket 10 would thus initially strike the coating 13 before contacting the bracket body 12 at that location. This is shown schematically in FIG. 3, in which rays of light 42 strike the coating 13. The majority of incident rays 42 do not reflect at the interface of the coating 13, which may be saliva coated, and so may pass through the coating 13 to the bracket body 12. The orthodontic bracket 10 is therefore less likely to be seen by others in the vicinity of the patient.

With continued reference to FIGS. 1 and 2, the lingual side 20 of the bracket body 12 is configured to be secured to the tooth in any conventional manner, such as by an appropriate orthodontic cement or adhesive or by a band around an adjacent tooth. The lingual side 20 may further be provided with a pad 32 defining a bonding base that is secured to the surface of the tooth. In one embodiment, the coating 13 may be on the pad 32 and so be less visible.

As shown in FIGS. 1 and 2, the bracket body 12 includes a base surface 34 and a pair of opposed slot surfaces 36, 38 projecting labially from the base surface 34 that collectively define the archwire slot 16 extending in a mesial-distal direction from mesial side 26 to distal side 28. The slot surfaces 36, 38 and base surface 34 are substantially encapsulated or embedded within the material of the bracket body 12 and, in one embodiment, may be defined by the coating 13. The coating 13 may therefore contact the archwire 18 during treatment. The archwire slot 16 of the bracket body 12 may be designed to receive the archwire 18 in any suitable manner.

In one embodiment, the bracket body 12 and/or the ligating slide 14 is formed from a ceramic material. For example, the ceramic material may include polycrystalline or monocrystalline ceramic materials that are transparent or translucent in the visible light spectrum (i.e., wavelengths from about 390 nm to about 700 nm). By way of example and not limitation, the ceramic material may be polycrystalline ceramic, such as those described in commonly owned U.S. Pat. No. 8,585,398, which is incorporated by reference herein in its entirety. In one embodiment, the polycrystalline ceramic is polycrystalline alumina. Alternatively, the ceramic material may be monocrystalline, such as single crystal alumina. Other suitable materials include, without limitation, polycarbonate, acrylic, plastic, resin, and metals such as titanium, stainless steel, and nickel-free metals.

As is described above, a portion of the bracket body 12 or the entire surface of the bracket body 12 may be coated so as to decrease the visibility of the bracket body 12. By way of example, with reference to FIGS. 3 and 4, the nano-pillar coating 13 may be an array of nano sized (about 10 nm or less in diameter) pillar-like structures 44 having an overall conical shape. That is, the individual pillar structures 44 may taper to a point 46 at one end and terminate at a wider base 48 in the direction of the bracket body 12. By way of example only, the structure 44 may be about 60 nm in diameter near the point 46 and about 130 nm in diameter near the base 48. In view of the structure shown in FIG. 4, the nano-pillar coating 13 may not include nano rods having a generally uniform thickness top to bottom. The nano-pillar coating 13 may exhibit a negative index of refraction at wavelengths in the visible light spectrum.

With reference to FIG. 3, while the thickness of the nano-pillar coating 13 is not thought to be limiting, the coating 13 may be a few micrometers thick and, for example, range in thickness from about 2 μm to about 5 μm. The coating 13 may be deposited or grown following injection molding the bracket body 12 or otherwise formed on the bracket body 12 during injection molding. Further, the coating 13 may be formed on the bracket body 12 and may be subjected to a deep reaction ion etching to form the nano-pillars. The materials for the coating 13 may include, without limitation, plastics, ceramics, and silicone.

The nano-pillar coating 13 improves the aesthetic appearance of the orthodontic bracket 10 by absorbing and/or transmitting, rather than reflecting, incident visible light, thus making the orthodontic bracket 10 significantly less noticeable. It is believed that the coating 13 has anti-reflective properties so that light that impinges upon the coating 13 is not reflected outwardly. The nano-pillar coating 13 may absorb up to 99% of incident light, which allows the sides 20, 22, 24, 26, 28, 30 (particularly the labial side 30) to blend into the background of the patient's tooth. In this way, the coating 13 can reduce the reflectivity, particularly the reflectivity of flat surfaces. Therefore, the nano-pillar coating 13 may nearly eliminate light reflecting off of the orthodontic bracket 10.

In an aspect of the present invention, the nano-pillar coating 13 described herein advantageously provides a more efficient medium to activate photo-initiators in light-activated orthodontic adhesives used to temporarily attach the orthodontic bracket 10 to the surface of a patient's tooth. In that regard, it is important to bond orthodontic appliances with a high bonding integrity as that translates to the durability and longevity of the bond. A significant factor in attaining high bonding integrity is a quick, high-strength cure of the adhesive. In one embodiment, due to the ability of the nano-pillar coating 13 to transmit almost all of the light impinging the surface of the orthodontic bracket 10, the coating 13 provides a more efficient medium to activate the photo-initiators in light activated orthodontic adhesives (i.e., where the bracket body 12 also transmits light to the surface of the patient's tooth).

Further, the nano-pillar coating 13 may act as an anti-microbial barrier coating. Advantageously, this prevents bacteria from attaching to a surface of the orthodontic bracket 10 and so not only improves the aesthetic properties, but retards calculus build-up. Further, the nano-pillar coating 13 may minimize or prevent bacteria from attaching to the orthodontic bracket 10. This benefit can reduce the risk of failure and may promote better long-term functionality (i.e., improved reliability) of the self-ligating mechanism (e.g., the ligating slide 14).

In one aspect of the present invention, the nano-pillar coating 13 forms each of the opposed slot surfaces 36, 38 and the base surface 34 of the archwire slot 16. The archwire 18 therefore contacts the coating 13, and not the bracket body 12, during orthodontic treatment. Contact with the coating 13 may decrease friction between the archwire 18 and the archwire slot 16. In order for a malocclusion to be corrected, the archwire 18 must return to its original, ideal shape. To return to this ideal shape, the archwire 18 must slide relative to the base surface 34 and opposed slot surfaces 36, 38 of the archwire slot 16. Friction between the archwire 18 and the archwire slot 16 can slow down this sliding process and so reduce the rate of tooth movement. The nano-pillar coating 13 may reduce the contact friction. Orthodontic treatment may proceed more quickly. In one embodiment, the nano-pillar coating 13 is less rough than a non-coated surface of the bracket body 12.

In addition to or alternatively, the nano-pillar coating 13 may reduce notching of the archwire 18. In that regard, when a patient chews food, the archwire 18 may rub against the bracket body, particularly against one or more corners of the archwire slot. In cases where an archwire is relatively soft compared to an orthodontic bracket, a rubbing or chafing motion has been shown to cause notching of the archwire. In one embodiment, by reducing the friction between the archwire 18 and the archwire slot 16, via the presence of the coating 13, the archwire 18 is less likely to be notched.

In one embodiment, the ligating slide 14 may also be formed from a ceramic material having the nano-pillar coating 13 on the external surfaces (e.g., the labial-most surface of the ligating slide 14). In one embodiment, the ceramic material may be the same as that used to form the bracket body 12, although embodiments of the invention are not so limited. Those of ordinary skill in the art will recognize, however, other suitable materials that provide an aesthetic appearance to the ligating slide 14 may be used and coated with the nano-pillar coating 13. Forming both the bracket body 12 and ligating slide 14 from a ceramic material having a nano-pillar coating 13 will reduce the visibility of the orthodontic bracket 10 and consequently further improve aesthetics of self-ligating orthodontic brackets during use.

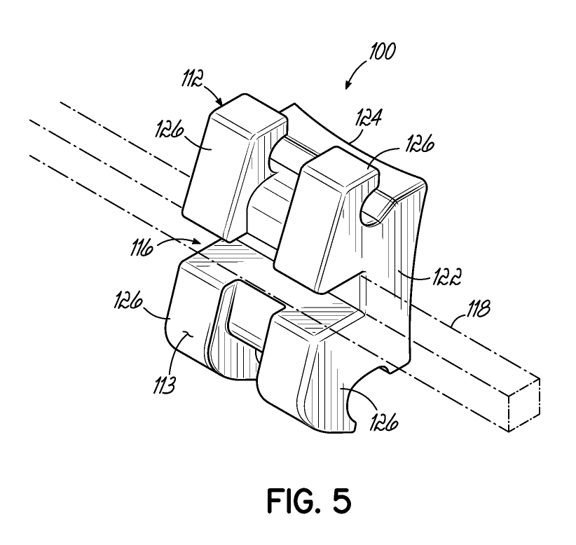

While the above embodiments of the invention are described with reference to self-ligating orthodontic brackets, non-self-ligating brackets are also contemplated. For example, with reference to FIG. 5, in one embodiment, the orthodontic appliance is a twin tie-wing orthodontic bracket 100 in which ligation of an archwire may be achieved with ligatures. The twin tie-wing orthodontic bracket 100 includes a bracket body 112 and a nano-pillar coating 113, described above, which covers all or a majority of the visible portions of the bracket body 112. The bracket body 112 has an archwire slot 116 that receives an archwire 118 (shown in phantom) for applying corrective forces to the teeth during orthodontic treatment. The bracket body 112 includes a base portion 122 including a bonding base surface 124 designed to be secured to a tooth in any conventional manner, for example, by an appropriate orthodontic cement/adhesive. The bracket body 112 further includes a pair of orthodontic tie-wings 126 which extend from the bracket body 112 and overhang the base portion 122 to allow proper ligation of an orthodontic archwire in the archwire slot 116.

While the present invention has been illustrated by a description of various embodiments and while these embodiments have been described in some detail, it is not the intention of the inventors to restrict or in any way limit the scope of the appended claims to such detail. Thus, additional advantages and modifications will readily appear to those of ordinary skill in the art. The various features of the invention may be used alone or in any combination depending on the needs and preferences of the user.

What is claimed is:

1. An orthodontic appliance comprising:
a nano-pillar coating on one or more external surfaces that are visible during use of the orthodontic appliance.

2. The orthodontic appliance of claim 1, wherein the nano-pillar coating is configured to absorb 99% of the incident visible light.

3. The orthodontic appliance of claim 1, wherein the nano-pillar coating has a pillar-like structure that includes a plurality of pillar structures, each pillar structure including a point that widens to a base.

4. The orthodontic appliance of claim 1, wherein the nano-pillar coating has a negative index of refraction at wavelengths in the visible light spectrum.

5. The orthodontic appliance of claim 1, wherein the nano-pillar coating does not include nano rods.

6. An orthodontic bracket comprising:
a bracket body that includes an archwire slot, and
a nano-pillar coating on one or more external surfaces of the bracket body that are visible during use of the orthodontic bracket.

7. The orthodontic bracket of claim 6, wherein the nano-pillar coating is configured to absorb 99% of the incident light.

8. The orthodontic bracket of claim 6, wherein the nano-pillar coating has a pillar-like structure that includes a plurality of pillar structures, each pillar structure including a point that widens to a base.

9. The orthodontic bracket of claim 6, wherein the nano-pillar coating has a negative index of refraction at wavelengths in the visible light spectrum.

10. The orthodontic bracket of claim 6, wherein the nano-pillar coating does not include nano rods.

11. The orthodontic bracket of claim 6, further comprising:
a movable member that is engaged with the bracket body and is movable between an opened position and a closed position, and
the nano-pillar coating on one or more external surfaces of the movable member.

12. The orthodontic bracket of claim 6, wherein the bracket body is a ceramic material.

13. The orthodontic bracket of claim 6, wherein the ceramic material is transparent or translucent.

14. A method of making an orthodontic bracket including a bracket body having an archwire slot, the method comprising:
forming a nano-pillar coating on at least a portion of the bracket body.

15. The method of claim 14, wherein the orthodontic bracket further includes a movable member and wherein forming includes forming the nano-pillar coating on the movable member.

16. The method of claim 14, further including injection molding the bracket body and wherein forming the nano-pillar coating occurs after injection molding.

17. The method of claim 14, further including injection molding the bracket body and wherein forming the nano-pillar coating occurs during injection molding.
